(19) United States
(12) Patent Application Publication
(10) Pub. No.: US 2026/0077001 A1
(43) Pub. Date: Mar. 19, 2026

(54) BACTERIAL COMPOSITIONS AND METHODS OF USE

(71) Applicant: University of Florida Research Foundation, Inc, Gainesville, FL (US)

(72) Inventors: Christian Jobin, Gainesville, FL (US); Rachel Newsome, Gainesville, FL (US)

(21) Appl. No.: 19/107,629

(22) PCT Filed: Sep. 1, 2023
(86) PCT No.: PCT/US2023/031871

§ 371 Date: Feb. 28, 2025

Related U.S. Application Data
(60) Provisional application No. 63/403,644, filed on Sep. 2, 2022.
(60) Provisional application No. 63/417,833, filed on Oct. 20, 2022.
(60) Provisional application No. 63/432,426, filed on Dec. 14, 2022.

(51) Int. Cl. A61K 35/741; A61K 35/00; A61K 39/395; C07K 16/28; C12N 1/20; C12R 1/01

(52) U.S. Cl. CPC A61K 35/741; A61K 39/39541; C07K 16/2818; C12N 1/205; A61K 2035/115; C12R 2001/01

(57) ABSTRACT

Aspects of the disclosure relate to compositions and methods for modulating the immune response of a subject. The disclosure is based, in part, on compositions comprising a bacterial consortium that promotes interferon (IFN) response in the gut microbiome of a subject and enhances the anti-tumor effects of certain immune checkpoint inhibitor (ICI) therapies. In some embodiments, the compositions are useful for treating a subject having cancer.

### CROSS REFERENCE TO RELATED APPLICATIONS AND INCORPORATION BY REFERENCE

This application claims the benefit of U.S. Provisional Patent Application No. 63/432,426, filed Dec. 14, 2022; U.S. Provisional Patent Application No. 63/417,833 , filed Oct. 20, 2022; and U.S. Provisional Patent Application No. 63/403,644 , filed Sep. 2, 2022, each of which is hereby incorporated by reference in their entireties.

### FIELD

The invention relates to bacterial compositions and use thereof.

### BACKGROUND

Lung cancer remains the leading cause of cancer death among people in the United States. The field of cancer research has been particularly attentive to the interaction between bacteria and therapeutics. Immune checkpoint inhibitors (ICI) can be highly effective in the treatment of non-small cell lung cancer (NSCLC), but only ˜20-30% of patients experience a complete response.

### SUMMARY

The disclosure provides a composition comprising an isolated population of Bacteroides ovatus bacterial cells comprising three or more different Bacteroides ovatus subspecies; an isolated population of Bacteroides intestinalis bacterial cells comprising two or more different Bacteroides intestinalis subspecies; and an isolated population of Bacteroides vulgatus bacterial cells. In some aspects, an isolated population of Bacteroides ovatus bacterial cells comprises Bacteroides ovatus M15A, Bacteroides ovatus M1B7, and Bacteroides ovatus M2H3 bacterial cells. In some aspects, an isolated population of Bacteroides intestinalis bacterial cells comprises Bacteroides intestinalis M2C7 and Bacteroides intestinalis M2F3 bacterial cells. In some aspects, an isolated population of Bacteroides vulgatus comprises Bacteroides vulgatus M2E6 bacterial cells.

In some aspects, a composition comprises between 1×10¹ and 1×10¹⁰ colony forming units (CFU) of bacteria. In some embodiments, a composition comprises about 1×10¹, 1×10², 1×10³, 1×10⁴, 1×10⁵, 1×10⁶, 1×10⁷, 1×10⁸, 1×10⁹, or 1×10¹⁰ CFU of bacteria. In some aspects, an isolated population of Bacteroides ovatus bacterial cells comprises between about 1.5×10⁴ and 1.5×10¹⁰ colony forming units (CFU). In some aspects, an isolated population of Bacteroides intestinalis bacterial cells comprises between about 1.5×10⁴ and 1.5×10¹⁰ colony forming units (CFU). In some aspects, an isolated population of Bacteroides vulgatus bacterial cells comprises between about 1.5×10⁴ and 1.5×10¹⁰ colony forming units (CFU).

In some aspects, a ratio of the isolated population of Bacteroides ovatus to the isolated population of Bacteroides intestinalis to the isolated population of Bacteroides vulgatus ranges from about 10:1:1 to about 1:10:1 to about 1:1:10 CFU. In some embodiments, a ratio of the isolated population of Bacteroides ovatus to the isolated population of Bacteroides intestinalis to the isolated population of Bacteroides vulgatus is about 1:1:1 CFU.

In some embodiments, a composition further comprises one or more pharmaceutically acceptable excipients. In some embodiments, a composition does not contain fecal matter. In some embodiments, a composition comprises fecal matter.

In some aspects, the disclosure provides a method for increasing interferon (IFN) (e.g., IFNγ) secretion in a subject, the method comprising administering to the subject a composition as described herein. The disclosure also provides a method for treating cancer in a subject, the method comprising administering to the subject a composition as described herein. In some aspects, a subject is a mammal, such as a human. In some aspects, a subject has or is suspected of having cancer (e.g., non-small cell lung carcinoma, melanoma, breast cancer, gastric cancer, prostate cancer, renal cancer, etc.). In some embodiments, cancer is non-small cell lung carcinoma (NSCLC). Optionally, the method further comprises administering an immune checkpoint inhibitor (ICI) to the subject. In some embodiments, the ICI comprises an anti-PD-1 antibody.

The disclosure further provides use of a composition disclosed herein to increase interferon (e.g., IFNγ) production in a subject or treat cancer in a subject in need thereof. The disclosure also contemplates use of a composition disclosed herein in the preparation of a medicament for increasing interferon (e.g., IFNγ) production in a subject or treating cancer in a subject in need thereof. Also provided is a composition disclosed herein for use in increasing interferon (e.g., IFNγ) production in a subject or treating cancer in a subject in need thereof.

Additional features and variations of the materials and methods of the disclosure will be apparent to those skilled in the art from the entirety of this application, including the figures and detailed description, and all such features are intended as aspects of the invention. Features of the invention described herein can be re-combined into additional embodiments that also are intended as aspects of the invention, irrespective of whether the combination of features is specified as an aspect or embodiment of the invention. The entire document is intended to be related as a unified disclosure, and it should be understood that all combinations of features described herein (even if described in separate sections) are contemplated, even if the combination of features is not found together in the same sentence, or paragraph, or section of this document. Also, only such limitations which are described herein as critical to the invention should be viewed as such; variations of the invention lacking limitations which have not been described herein as critical are intended as aspects of the invention.

### BRIEF DESCRIPTION OF DRAWINGS

FIGS. 1A-1D show representative data indicating that responder microbiota transplantation decreases tumor growth compared to non-responder colonized mice following immunotherapy treatment. FIG. 1A shows a growth curve of Lewis Lung Carcinoma (LLC)-luc subcutaneous xenograft tumors after human fecal microbiota transplant from responder (n=4) or non-responder (n=6) pooled feces into germ-free mice (n=9/group) treated with anti-PD-1 monoclonal antibody injection. P=0.023 at endpoint (ANOVA). (Tumor volume=y-axis; days post LLC implantation=x-axis; square=R feces; circle=NR feces) FIG. 1B shows a growth curve of LLC-luc subcutaneous xenograft tumors after human fecal microbiota transplant from responder (n=4) or non-responder (n=6) pooled feces into germ-free mice (n=5/group). P>0.05 at endpoint (ANOVA). (Tumor volume=y-axis; days post LLC implantation=x-axis; carat=R feces; triangle=NR feces) FIG. 1C shows the mean±SEM of tumor weight at endpoint for mice treated with anti-PD-1 monoclonal antibody. P=0.033 (Mann Whitney test). FIG. 1D shows the mean±SEM of tumor weight at endpoint for untreated mice. P>0.05 (Mann Whitney test). Weight of tumor in grams is depicted on the y-axis.

FIG. 2 shows representative workflow for high-throughput microbial isolation from feces of responder associated (R-associated) mice. The fecal sample is aliquoted and homogenized in anaerobic MEGA media to a final suspension of 10% (w/v). The fecal sample is then diluted to a theoretical target loading of 0.3 cells/well in addition to 50 μM of resorufin as an anaerobic growth indicator. The diluted sample is then vacuum loaded onto 6,000 nanowell growth chambers, sealed and imaged. Wells likely to contain culturable, single bacterial isolates, are identified and sterile transfers are performed from the array into 96 well plates from which glycerol stocks can be prepared, cataloged and stored at −80° C. in sealed gas packs. To identify individual isolates, individual bacterial cultures undergo Biotyper analysis. Those isolates that do not receive an identification from the Biotyper can then have genomic DNA extracted for full length 16S PCR and Sanger sequencing by a reference laboratory. This is an exemplary method of obtaining bacteria described herein.

FIG. 3 shows representative workflow for an assay for immunomodulatory effects (IFNγ secretion) of cell free supernatant from individual bacteria. Isolates are cultured in 96-well deep well plates for 3 days under anaerobic conditions in MEGA media. Cell-free supernatant is obtained by filtering cultures by 0.22 μM syringe filtration. Cell-free supernatant is added to the serum-free culture media of primary mouse splenic CD8+T cells at a ratio of 1:100, followed by six hours of incubation. After incubation, cells are harvested and stained for flow cytometric analysis, which is performed on the BD Fortessa LSR, followed by analysis using FlowJo.

FIG. 4 shows a schematic depicting testing for Responder consortium (“R consortium” or “six-consortium”) anti-PD-1 mediated anti-tumor effect. Germ free mice were colonized with 1×10⁷ colony forming units (CFU) of six-consortium, R feces or non-responder (NR) feces for 2 weeks followed by implantation with 1×10⁶ Lewis Lung Carcinoma (LLC) xenograft tumor cells. Mice then underwent four rounds of anti-PD-1 therapy by intraperitoneal injection, with tumors being measured every three days by manual caliper. At endpoint tumors were harvested for flow cytometric analysis.

FIG. 5 shows representative data indicating the that six-consortium decreases tumor growth compared to non-responder (NR) colonized xenograft tumors after human fecal microbiota transplant from six-consortium, R feces, or NR pooled feces into germ-free mice treated with anti-PD-1 monoclonal antibody injection. P=0.0046 between R feces and NR feces and P<0.0001 between six-consortium and NR feces at endpoint (ANOVA). (Tumor volume=y-axis; days post LLC implantation=x-axis; circle=six-consortium; square=R feces; triangle=NR feces)

FIG. 6 shows a schematic depicting testing the effect of anti-IFNγ depletion on six-consortium-, anti-PD-1-mediated anti-tumor effect. Germ-free mice were colonized with 1×10⁷ CFU of six-consortium or NR feces for 2 weeks followed by implantation with 1×10⁶ Lewis Lung Carcinoma (LLC) xenograft tumor cells. Mice then underwent four rounds of anti-PD-1 therapy with or without every other day anti-IFNγtherapy, both by intraperitoneal injection, with tumors being measured every three days by manual caliper. At endpoint, tumors were harvested for flow cytometric analysis.

FIGS. 7A-7B show representative data indicating in vivo depletion of IFNγ abrogates beneficial effect of responder consortium on anti-PD-1. Growth curve of LLC-luc subcutaneous xenograft tumors after human fecal microbiota transplant from responder consortium (also referred to herein as “six-consortium,” “six-consort,” or “6-consort”) or non-responder pooled feces into germ-free mice treated with anti-PD-1 or combination anti-PD-1/anti-IFNγ monoclonal antibody injection. P=0.041 between six-consortium and NR feces and P=0.012 between six-consortium and six-consortium with anti-IFNγ depletion (ANOVA). No significance between NR feces and NR feces with anti-IFNγ depletion. (Tumor volume=y-axis; days post LLC implantation=x-axis; * anti-IFNγ; circle=six-consortium anti-PD-1; square=R consortium anti-PD-1 and anti-IFNγ; triangle=NR feces anti-PD-1; X=NR feces anti-PD-1 and anti-IFNγ)

FIGS. 8A-8B shows representative data indicating that the six-consortium enhances anti-PD-1 treatment in vivo through IFNγ. Germ-free LLC tumor-bearing mice were colonized with the six-consort or the pooled NR patient samples (10⁷ CFU) and treated with anti-PD-1 or a combination of anti-PD-1/anti-IFNγ antibodies. Six-consort-treated mice treated with only anti-PD-1 showed increased serum IFNγ concentration (FIG. 8A) and tumor infiltrating cytotoxic IFNγ+CD8+T cells (FIG. 8B) compared to IFNy-depleted mice treated with the six-consortium. FIG. 8A shows mean±SEM of serum IFNγ concentration in pg/mL 20 days post treatment. P values calculated by Mann-Whitney U test. FIG. 8B shows mean±SEM of intratumoral IFNγ+CD8+frequency of resected subcutaneous allograft tumors from human microbiota colonized non-responder mice with or without anti-IFNγ depletion (n=4 and n=6, respectively) and six-consort-treated mice with or without IFNγ depletion (n=5 and n=6, respectively). P values calculated by Mann-Whitney U test.

FIGS. 9A-9F are MALDI-TOF spectra for Bacteroides ovatus M15A (FIG. 9A), Bacteroides ovatus M1B7 (FIG. 9B), Bacteroides ovatus M2H3 (FIG. 9C), Bacteroides intestinalis M2C7 (FIG. 9D), Bacteroides intestinalis M2F3 (FIG. 9E), and Bacteroides vulgatus M2E6 (FIG. 9F) generated using a MALDI Biotyper® (Bruker).

### DETAILED DESCRIPTION

The disclosure relates, in various aspects, to compositions and methods for modulating the immune response of a subject. For example, aspects of the disclosure are based, at least in part, on compositions comprising a bacterial consortium (e.g., a composition comprising one or more isolated populations of bacteria) that promotes interferon gamma (IFNγ) response in the gut microbiome of a subject.

In various aspects, the disclosure provides a composition comprising an isolated population of Bacteroides ovatus bacterial cells, an isolated population of Bacteroides intestinalis bacterial cells, and/or an isolated population of Bacteroides vulgatus bacterial cells. In various aspects, the composition comprises two more species of the Bacteroides bacterial cells, e.g., Bacteroides ovatus bacterial cells and Bacteroides intestinalis bacterial cells, Bacteroides ovatus bacterial cells and Bacteroides vulgatus bacterial cells, or Bacteroides intestinalis bacterial cells and Bacteroides vulgatus bacterial cells. In various aspects, the composition comprises a population of Bacteroides ovatus bacterial cells, a population of Bacteroides intestinalis bacterial cells, and population of Bacteroides vulgatus bacterial cells.

Optionally, the composition comprises multiple subspecies within the Bacteroides ovatus, Bacteroides intestinalis, and/or Bacteroides vulgatus species. For instance, the population of Bacteroides ovatus bacterial cells may comprise one, two, three, four, five or more different Bacteroides ovatus subspecies (e.g., the population of Bacteroides ovatus bacterial cells comprises three or more different Bacteroides ovatus subspecies, such as three different Bacteroides ovatus subspecies). In this regard, the disclosure contemplates an isolated population of Bacteroides ovatus bacterial cells comprising Bacteroides ovatus M15A, Bacteroides ovatus M1B7, and/or Bacteroides ovatus M2H3. Optionally, an isolated population of Bacteroides ovatus bacterial cells comprises Bacteroides ovatus M15A, Bacteroides ovatus M1B7, and Bacteroides ovatus M2H3. The population of Bacteroides intestinalis bacterial cells may comprise one, two, three, four, five or more different Bacteroides intestinalis subspecies (e.g., the population of Bacteroides intestinalis bacterial cells comprises two or more different Bacteroides ovatus subspecies, such as two different Bacteroides intestinalis subspecies). In this regard, the disclosure contemplates an isolated population of Bacteroides intestinalis bacterial cells comprising Bacteroides intestinalis M2C7 and/or Bacteroides intestinalis M2F3, such as a population comprising Bacteroides intestinalis M2C7 and Bacteroides intestinalis M2F3. The population of Bacteroides vulgatus bacterial cells optionally comprises one, two, three, four, five or more different Bacteroides vulgatus subspecies (e.g., the population of Bacteroides vulgatus bacterial cells comprises one or more different Bacteroides ovatus subspecies, such as one Bacteroides vulgatus subspecies). Optionally, the isolated population of Bacteroides vulgatus comprises Bacteroides vulgatus M2E6. In various aspects, the disclosure provides a composition comprising (i) an isolated population of Bacteroides ovatus bacterial cells comprising three or more different Bacteroides ovatus subspecies (e.g., Bacteroides ovatus M15A, Bacteroides ovatus M1B7, and Bacteroides ovatus M2H3); (ii) an isolated population of Bacteroides intestinalis bacterial cells comprising two or more different Bacteroides intestinalis subspecies (e.g., Bacteroides intestinalis M2C7 and Bacteroides intestinalis M2F3); and (iii) an isolated population of Bacteroides vulgatus bacterial cells (e.g., Bacteroides vulgatus M2E6).

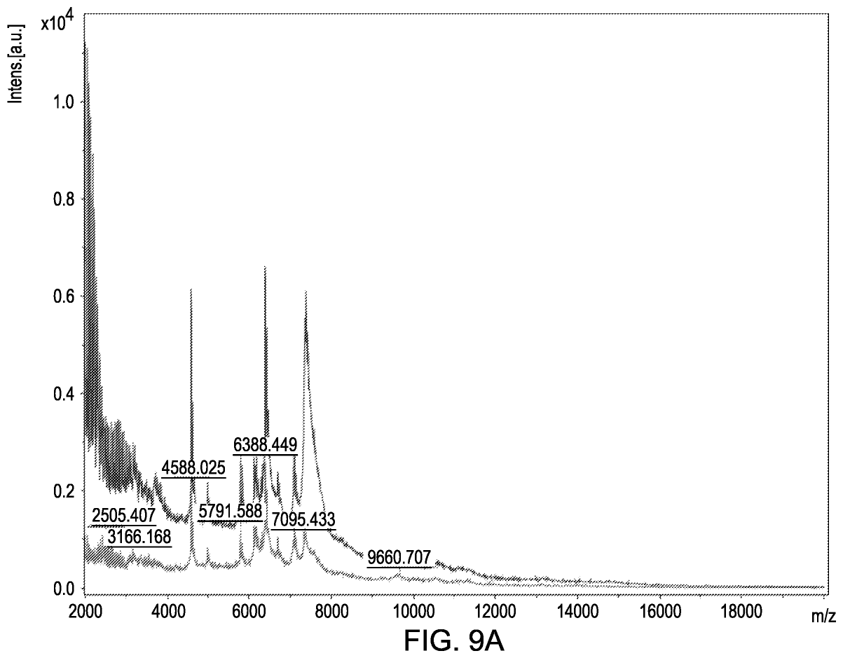

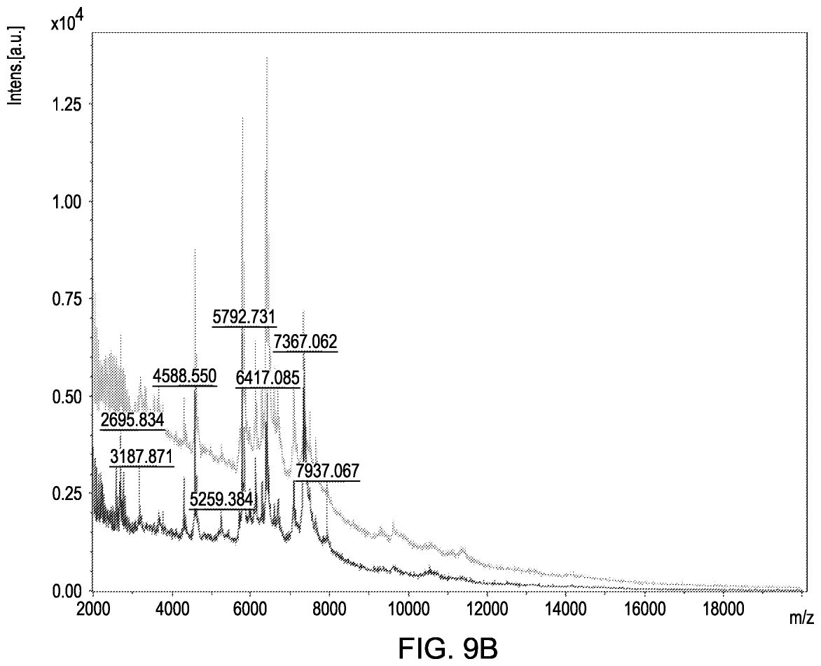

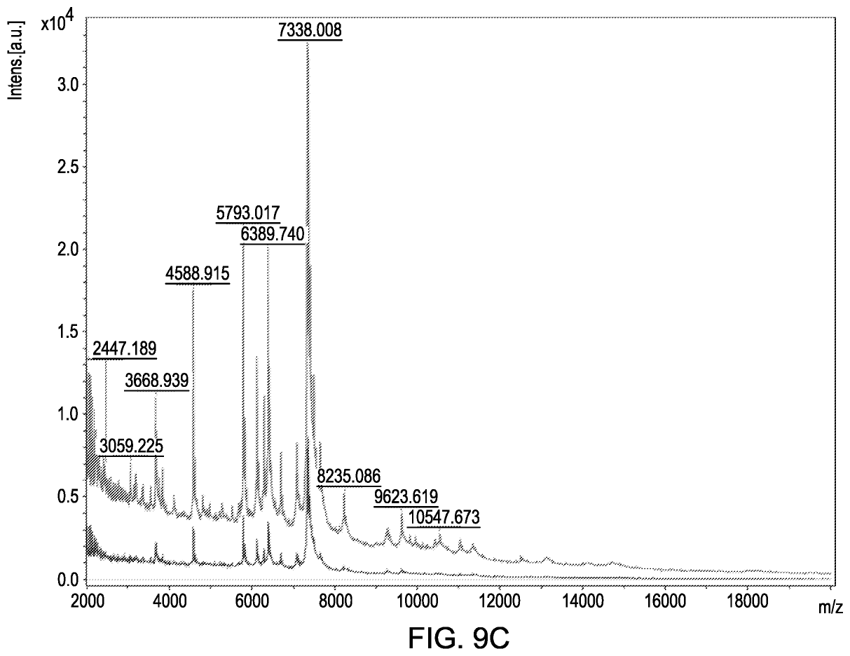

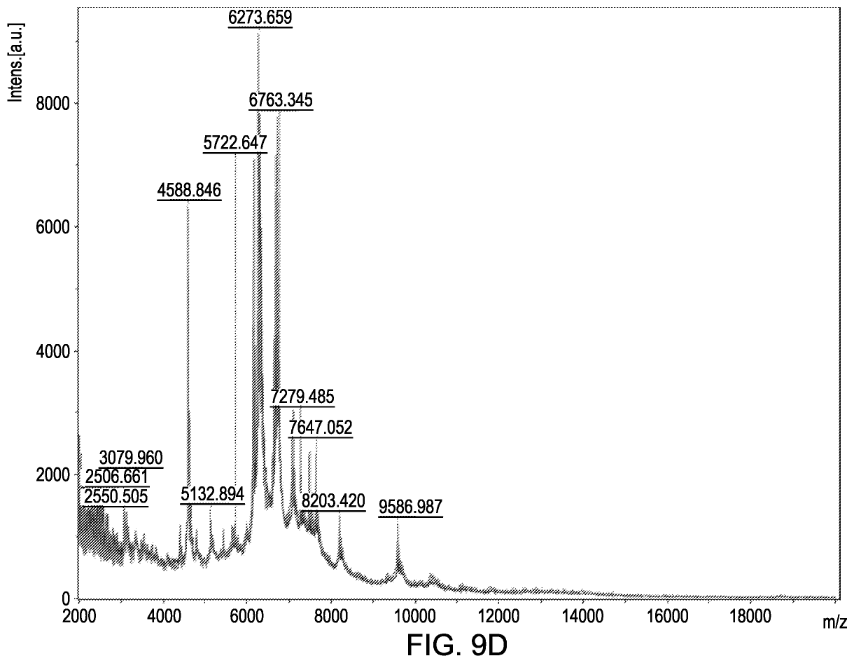

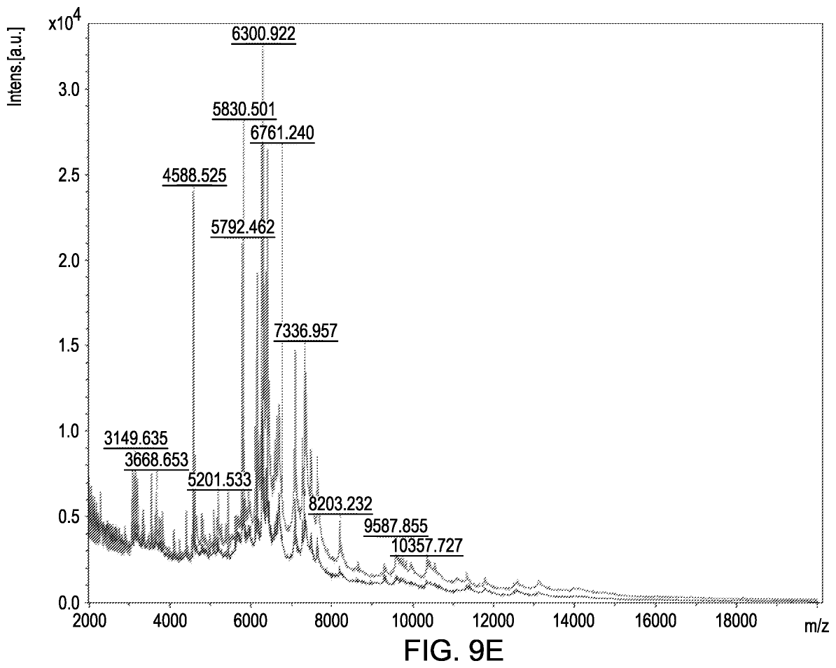

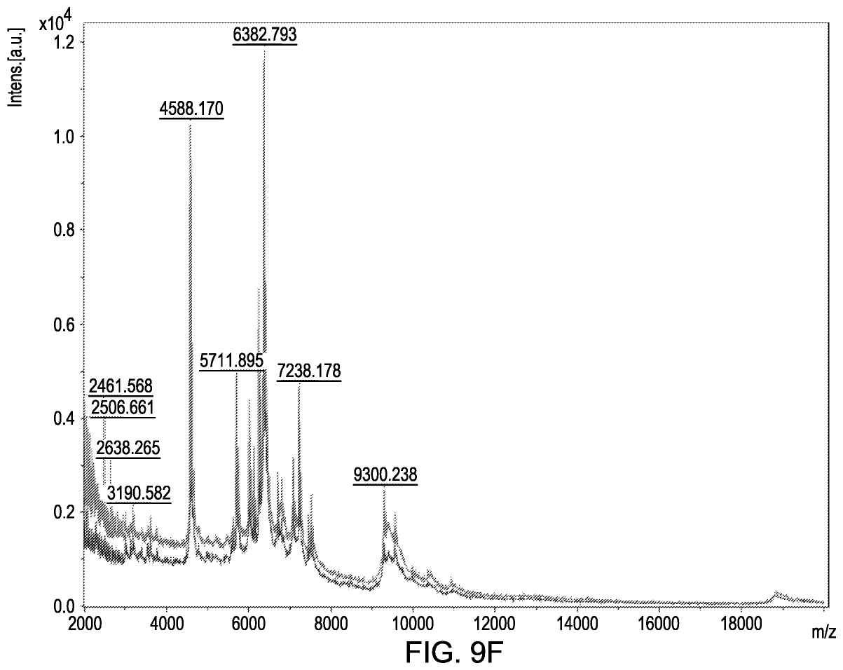

MALDI-TOF spectra generated using a MALDI Biotyper® (Bruker) are provided in FIGS. 9A-9F for Bacteroides ovatus M15A (FIG. 9A), Bacteroides ovatus M1B7 (FIG. 9B), Bacteroides ovatus M2H3 (FIG. 9C), Bacteroides intestinalis M2C7 (FIG. 9D), Bacteroides intestinalis M2F3 (FIG. 9E), and Bacteroides vulgatus M2E6 (FIG. 9F). MALDI-TOF (Matrix-Assisted Laser Desorption/Ionization Time-of-Flight) mass spectrometry measures highly abundant proteins found in microorganisms and generates a unique “fingerprint” of an organism based on abundance of these proteins and differences found between organisms. A representative method for characterizing bacteria using MALDI-TOF spectra is provided in Example 3. Bacteria described herein are characterized by (i) a MALDI-TOF spectrum having at least a first peak at 2505.407 m/z, a second peak at 3166.168 m/z, a third peak at 4588.025 m/z, a fourth peak at 5791.588 m/z, a fifth peak at 6388.449 m/z, a sixth peak at 7095.43 m/z 3, and a seventh peak at 9660.707 m/z (Bacteroides ovatus M15A (FIG. 9A)); (ii) a MALDI-TOF spectrum having at least a first peak at 2695.834 m/z, a second peak at 3187.871 m/z, a third peak at 4588.550 m/z, a fourth peak at 5259.384 m/z, a fifth peak at 5792.731 m/z, a sixth peak at 6417.085 m/z, a seventh peak at 7367.062 m/z, and an eighth peak at 7937.067 m/z (Bacteroides ovatus M1B7 (FIG. 9B)); (iii) a MALDI-TOF spectrum having at least a first peak at 2447.189 m/z, a second peak at 3059.225 m/z, a third peak at 3668.939 m/z, a fourth peak at 4588.915 m/z, a fifth peak at 5793.017 m/z, a sixth peak at 6389.740 m/z, a seventh peak at 7338.008 m/z, an eighth peak at 8235.086 m/z, a nineth peak at 9623.619 m/z, and a tenth peak at 10547.673 m/z (Bacteroides ovatus M2H3 (FIG. 9C)); (iv) a MALDI-TOF spectrum having at least a first peak at 2506.661 m/z, a second peak at 2550.505 m/z, a third peak at 3079.960 m/z, a fourth peak at 4588.846 m/z, a fifth peak at 5132.894 m/z, a sixth peak at 5722.647 m/z, a seventh peak at 6273.659 m/z, an eighth peak at 6763.345 m/z, a nineth peak at 7279.485 m/z, a tenth peak at 7647.052 m/z, an eleventh peak at 8203.420 m/z, and a twelfth peak at 9586.987 m/z (Bacteroides intestinalis M2C7 (FIG. 9D)); (v) a MALDI-TOF spectrum having at least a first peak at 3149.635 m/z, a second peak at 3668.653 m/z, a third peak at 4588.525 m/z, a fourth peak at 5201.533 m/z, a fifth peak at 5792.462 m/z, a sixth peak at 5830.501 m/z, a seventh peak at 6300.922 m/z, an eighth peak at 6761.240 m/z, a nineth peak at 7336.957 m/z, a tenth peak at 8203.232 m/z, an eleventh peak at 9587.855 m/z, and a twelfth peak at 10357.727 m/z (Bacteroides intestinalis M2F3 (FIG. 9E)); and (vi) a MALDI-TOF spectrum having at least a first peak at 2461.568 m/z, a second peak at 2506.661 m/z, a third peak at 2638.265 m/z, a fourth peak at 3190.582 m/z, a fifth peak at 4588.170 m/z, a sixth peak at 5711.895 m/z, a seventh peak at 6382.793 m/z, an eighth peak at 7238.178 m/z, and a nineth peak at 9300.238 m/z (Bacteroides vulgatus M2E6 (FIG. 9F)).

An “isolated” population of bacteria generally refers to a population of bacterial cells removed from their natural habitat (i.e., removed from the biological niche in which the bacterial cells are naturally found). “Isolated” populations of bacteria also generally refer to populations which consist essentially of a particular type (e.g., species) of bacteria (i.e., is a substantially homogenous population of a particular type (e.g., species) of bacteria). In some embodiments, an isolated population of bacteria comprises one or more (e.g., 1, 2, 3, 4, 5, 6, 7, 8, 9, 10, or more) subspecies of a particular bacterial species. For example, an isolated population of Bacteroides ovatus may comprise two, three, four, or more subspecies of Bacteroides ovatus. A composition may comprise multiple “isolated” populations of bacteria, wherein each population comprises a particular species of bacteria removed from its natural habitat and which is composed mostly (e.g., at least 70%, at least 75%, at least 80%, at least 85%, at least 90%, at least 95%, or 100%) of the particular species of bacteria.

The amount of bacteria present in the composition is optionally between 1×10¹ and 5×10¹⁰ colony forming units (CFU) of bacteria (or more). For instance, in various aspects, the composition comprises between 1×10¹ and 1×10¹⁰ CFU of bacteria. Optionally, the composition comprises an isolated population of Bacteroides ovatus bacterial cells in an amount of between about 1.5×10⁴ and about 1.5×10¹⁰ CFUs (e.g., about 1.5×10⁴ CFUs, about 1.5×10⁵ CFUs, about 1.5×10⁶ CFUs, about 1.5×10⁷ CFUs, about 1.5×10⁸ CFUs, about 1.5×10⁹ CFUs, or about 1.5×10¹⁰ CFUs, or any range comprising these values as endpoints), an isolated population of Bacteroides intestinalis bacterial cells in an amount of between about 1.5×10⁴ and 1.5×10¹⁰ CFU (e.g., about 1.5×10⁴ CFUs, about 1.5×10⁵ CFUs, about 1.5×10⁶ CFUs, about 1.5×10⁷ CFUs, about 1.5×10⁸ CFUs, about 1.5×10⁹ CFUs, or about 1.5×10¹⁰ CFUs, or any range comprising these values as endpoints), and/or an isolated population of Bacteroides vulgatus bacterial cells in an amount of between about 1.5×10⁴ and 1.5×10¹⁰ CFU (e.g., about 1.5×10⁴ CFUs, about 1.5×10⁵ CFUs, about 1.5×10⁶ CFUs, about 1.5×10⁷ CFUs, about 1.5×10⁸ CFUs, about 1.5×10⁹ CFUs, or about 1.5×10¹⁰ CFUs, or any range comprising these values as endpoints) (and any combination of amounts of Bacteroides ovatus, Bacteroides intestinalis, and/or Bacteroides vulgatus). The relative amounts of the different bacterial species may be adjusted. For instance, in various aspects, the ratio of the isolated population of Bacteroides ovatus to the isolated population of Bacteroides intestinalis to the isolated population of Bacteroides vulgatus ranges from about 10:1:1 to about 1:10:1 to about 1:1:10 CFU. Optionally, the ratio of the isolated population of Bacteroides ovatus to the isolated population of Bacteroides intestinalis to the isolated population of Bacteroides vulgatus is about 1:1:1 CFU.

In various aspects, the composition of the disclosure comprises the bacterial populations described herein and one or more pharmaceutically acceptable excipients or carriers. The phrase “pharmaceutically acceptable” refers to ligands, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The composition may take the form of, e.g., a powder, tablet, coated tablet, capsule, suppository, solution, slurry, syrup, juice, suspension, or emulsion. For tablets and capsules, an active ingredient, such as bacteria cell populations, may be combined with binders, lubricants, disintegrants, and/or colorants. Examples of binders include, but are not limited to, starch, gelatin, natural sugars, natural and synthetic gums such as acacia, tragacanth and sodium oleate, sodium stearate, magnesium stearate, sodium benzoate, sodium acetate, sodium chloride, and the like. Examples of disintegrants include, but are not limited to, starch, methyl cellulose, agar, bentonite, xanthan gum, and the like. Liquid formulations may include carriers such as saline, sterile water, Ringer's solution, buffered saline, dextrose solution, maltodextrin solution, glycerol, or ethanol.

The composition of the present disclosure may be administered to a subject via any suitable method to deliver the payload to the gut of the subject, such as enteral administration routes (e.g., oral, buccal, or rectal routes of administration) or administration directly to the GI tract. In various aspects, the composition comprises fecal matter, which may be employed in fecal transplant methodologies. In alternative aspects, the composition does not comprise fecal matter. Optionally, the composition is administered to a subject in need thereof via oral administration or via fecal transplantation. Preferably, after administration of a composition of the disclosure to a subject, the populations of Bacteroides ovatus, Bacteroides intestinalis, and/or Bacteroides vulgatus colonize the subject.

The disclosure further provides methods of using the composition described herein to, e.g., modulate the immune response in a subject in need thereof. For example, the disclosure provides a method for increasing interferon (IFN) secretion (e.g., IFNγ) in a subject. The method comprises administering to the subject a composition described herein. Methods for measuring IFN production in a subject are well known in the art. Optionally, the composition of the disclosure mediates at least about a 10%, at least about a 20%, at least about a 30%, at least about a 40%, at least about a 50%, at least about a 60%, at least about a 70%, at least about an 80%, or at least about a 90% increase in IFN production in a subject (e.g., as detected in a biological sample from the subject) in a clinically relevant timeframe. The level of IFN production is compared to, e.g., the level of IFN production observed in the subject prior to administration of the composition or compared to a biologically matched control population which is not administered the composition. In various aspects, after the administration of the composition, interferon (IFN) (e.g., IFNγ) secretion is increased in the subject relative to IFN secretion before the administration.

The disclosure further provides a method of treating cancer in a subject. The method comprises administering to a subject in need thereof a composition described herein. The cancer in some aspects is one selected from the group consisting of acute lymphocytic cancer, acute myeloid leukemia, alveolar rhabdomyosarcoma, bone cancer, brain cancer (e.g., glioma), breast cancer (e.g., triple negative breast cancer), cancer of the anus, anal canal, or anorectum, cancer of the eye, cancer of the intrahepatic bile duct, cancer of the joints, cancer of the head, neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, gastrointestinal cancer (e.g., gastrointestinal carcinoid tumor), Hodgkin lymphoma, endometrial or hepatocellular carcinoma, hypopharynx cancer, kidney cancer, larynx cancer, liver cancer, lung cancer (e.g., non-small cell lung cancer, bronchioloalveolar carcinoma), malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer. In particular aspects, the cancer is non-small cell lung cancer (NSCLC). In various aspects, the subject has a solid tumor.

In some embodiments, administration of the bacterial consortium (or a composition described herein) enhances the anti-tumor effects of an immune checkpoint inhibitor (ICI) therapy, for example anti-PD-1 antibodies. An “immune checkpoint inhibitor” or “ICI” is any agent (e.g., compound or molecule) that that decreases, blocks, inhibits, abrogates or interferes with the function of a protein of an immune checkpoint pathway. Proteins of the immune checkpoint pathway regulate immune responses and, in some instances, prevent T cells from attacking cancer cells. In various aspects, the protein of the immune checkpoint pathway is, for example, CTLA-4, PD-1, PD-L1, PD-L2, B7-H3, B7-H4, TIGIT, VISTA, LAG3, CD112 TIM3, BTLA, or co-stimulatory receptor ICOS, OX40, 41BB, or GITR. In various aspects, the ICI is a small molecule, an inhibitory nucleic acid, or an inhibitor polypeptide. In various aspects, the ICI is an antibody, antigen-binding antibody fragment, or an antibody protein product, that binds to and inhibits the function of the protein of the immune checkpoint pathway (e.g., CTLA-4, PD-1, PD-L1, PD-L2, B7-H3, B7-H4, TIGIT, VISTA, LAG3, CD112 TIM3, BTLA, or co-stimulatory receptor ICOS, OX40, 41BB, or GITR). Suitable ICIs which are antibodies, antigen-binding antibody fragments, or antibody-like protein products are known in the art and include, but are not limited to, ipilimumab (CTLA-4; Bristol Meyers Squibb), nivolumab (PD-1; Bristol Meyers Squibb), pembrolizumab (PD-1; Merck), atezolizumab (PD-L1; Genentech), avelumab (PD-L1; Merck), and durvalumab (PD-L1; Medimmune) (Wei et al., Cancer Discovery 8:1069-1086 (2018)). Other examples of ICIs include, but are not limited to, IMP321 (LAG3: Immuntep); BMS-986016 (LAG3; Bristol Meyers Squibb); IPH2101 (KIR; Innate Pharma); tremelimumab (CTLA-4; Medimmune); pidilizumab (PD-1; Medivation); MPDL3280A (PD-L1; Roche); MEDI4736 (PD-L1; AstraZeneca); MSB0010718C (PD-L1; EMD Serono); AUNP12 (PD-1; Aurigene); MGA271 (B7-H3: MacroGenics); and TSR-022 (TIM3; Tesaro).

In various aspects, the present disclosure further provides a method of treating a subject in need thereof by administering a composition of the disclosure and further administering one or more ICIs to the subject, such as any one or more of the ICIs described herein. The disclosure contemplates combination therapy comprising a composition described herein with one ICI, two ICIs (e.g., a PD-1 inhibitor and a PD-L1 inhibitor, or a PD-1 inhibitor and a CTLA inhibitor), three ICIs, or more.

In various instances, the ICI is a PD-1 inhibitor. “Programmed Death-1” (PD-1), also known as cluster of differentiation 279 (CD279), refers to an immunoinhibitory receptor belonging to the CD28 family. PD-1 is expressed on previously activated T cells in vivo, and binds to two ligands, PD-L1 and PD-L2. The human PD-1 sequence can be found under GenBank Accession No. U64863. For example, the PD-1 inhibitor may bind to and inhibit the function of PD-1, e.g., an anti-PD-1 antibody, antigen binding antibody fragment, or an antibody-like molecule. In various aspects, the PD-1 inhibitor is durvalumab, atezolizumab, or avelumab. In various aspects, the ICI is a PD-L2 inhibitor. For example, the PD-L2 inhibitor binds to and inhibits the function of PD-L2, e.g., an anti-PD-L2 antibody, antigen binding antibody fragment, or an antibody-like molecule. In various aspects, the ICI is a PD-L1 inhibitor. For example, the PD-L1 inhibitor binds to and inhibits the function of PD-L1, e.g., an anti-PD-L1 antibody, antigen binding antibody fragment, or an antibody-like molecule. Examples of PD-1 and PD-L1 inhibitors are described in, e.g., U.S. Pat. Nos. 7,488,802; 7,943,743; 8,008,449; 8,168,757; 8,217,149: and PCT Patent Publication Nos. WO03042402, WO2008156712, WO2010089411, WO2010036959, WO2011066342, WO2011159877, WO2011082400, and WO2011161699; which are incorporated by reference herein in their entireties.

In various aspects, the ICI is an inhibitor of cytotoxic T-lymphocyte-associated protein 4. CTLA-4, also known as CD152, is a membrane protein expressed on T cells and regulatory T cells (Treg). CTLA-4 binds B7-1 (CD80) and B7-2 (CD86) on antigen-presenting cells (APC), which inhibits the adaptive immune response. In humans, CTLA-4 is encoded in various isoforms; an exemplary amino acid sequence is available as GenBank Accession No. NP_001032720. A representative anti-CTLA-4 antibody is ipilimumab (YERVOY®, Bristol-Myers Squibb).

The term “treat,” as well as words related thereto, do not necessarily imply 100% or complete treatment or remission. Rather, there are varying degrees of treatment of which one of ordinary skill in the art recognizes as having a potential benefit or therapeutic effect. In this respect, the methods of treating a disease of the present disclosure can provide any amount or any level of treatment. Furthermore, the treatment provided by the method may include treatment of one or more conditions or symptoms or signs of the disease being treated. For instance, the treatment method of the disclosure may inhibit one or more symptoms of the disease. Also, the treatment provided by the methods of the present disclosure may encompass slowing the progression of the disease. For example, the methods can treat cancer by virtue of enhancing the T cell activity or an immune response against the cancer, thereby reducing tumor or cancer growth, reducing metastasis of tumor cells, increasing cell death of tumor or cancer cells, and the like. Examples of a therapeutic response include (but are not limited to) one or more of the following improvements in the disease: (1) a reduction in the number of neoplastic cells; (2) an increase in neoplastic cell death; (3) inhibition of neoplastic cell survival; (4) inhibition (i.e., slowing to some extent, preferably halting) of tumor growth or appearance of new lesions; (5) decrease in tumor size or burden; (6) absence of clinically detectable disease, (7) decrease in levels of cancer markers; (8) an increased patient survival rate; and/or (9) some relief from one or more symptoms associated with the disease or condition (e.g., pain). For example, the efficacy of treatment may be determined by detecting of a change in tumor mass and/or volume after treatment. The size of a tumor may be compared to the initial size and dimensions as measured by CT, PET, mammogram, ultrasound, or palpation, as well as by caliper measurement or pathological examination of the tumor after biopsy or surgical resection. Response may be characterized quantitatively using, e.g., percentage change in tumor volume (e.g., the method of the disclosure results in a reduction of tumor volume by at least 10%, at least 20%, at least 30%, at least 40%, at least 50%, at least 60%, at least 70%, at least 80%, or at least 90%). Alternatively, tumor response or cancer response may be characterized in a qualitative fashion like “pathological complete response” (pCR), “clinical complete remission” (cCR), “clinical partial remission” (cPR), “clinical stable disease” (cSD), “clinical progressive disease” (cPD), or other qualitative criteria. In addition, treatment efficacy also can be characterized in terms of responsiveness to other immunotherapy treatment or chemotherapy. In various aspects, the methods of the disclosure further comprise monitoring treatment in the subject.

The subject of the methods described herein is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). In some aspects, the mammal is a human, optionally a human suffering from or suspected of suffering from cancer.

In any of the methods described herein, the composition may comprise an isolated population of Bacteroides ovatus bacterial cells, an isolated population of Bacteroides intestinalis bacterial cells, and/or an isolated population of Bacteroides vulgatus bacterial cells. A composition comprising one or two of the bacterial species is contemplated for use in the context of the methods. In various aspects, the composition of the methods comprises a consortium of all three bacterial species (a population of Bacteroides ovatus bacterial cells, a population of Bacteroides intestinalis bacterial cells, and population of Bacteroides vulgatus bacterial cells). As described herein, the composition optionally comprises multiple subspecies within the Bacteroides ovatus, Bacteroides intestinalis, and/or Bacteroides vulgatus species. In various aspects, the composition comprises (i) an isolated population of Bacteroides ovatus bacterial cells comprising three or more different Bacteroides ovatus subspecies (e.g., Bacteroides ovatus M15A, Bacteroides ovatus M1B7, and Bacteroides ovatus M2H3); (ii) an isolated population of Bacteroides intestinalis bacterial cells comprising two or more different Bacteroides intestinalis subspecies (e.g., Bacteroides intestinalis M2C7 and Bacteroides intestinalis M2F3); and (iii) an isolated population of Bacteroides vulgatus bacterial cells (e.g., Bacteroides vulgatus M2E6).

The amount of bacteria administered to a subject in need thereof is an amount effective to achieve a desired biological effect in a clinically relevant time period. For instance, a dose of bacteria administered to the subject is optionally between 1×10¹ and 5×10¹⁰ colony forming units (CFU) of bacteria (or more). For instance, in various aspects, a dose comprises between 1×10¹ and 1×10¹⁰ CFU of bacteria. Optionally, a dose comprises an isolated population of Bacteroides ovatus bacterial cells in an amount of between about 1.5×10⁴ and about 1.5×10¹⁰ CFUs (e.g., about 1.5×10⁴ CFUs, about 1.5×10⁵ CFUs, about 1.5×10⁶ CFUs, about 1.5×10⁷ CFUs, about 1.5×10⁸ CFUs, about 1.5×10⁹ CFUs, or about 1.5×10¹⁰ CFUs, or any range comprising these values as endpoints), an isolated population of Bacteroides intestinalis bacterial cells in an amount of between about 1.5×10⁴ and 1.5×10¹⁰ CFU (e.g., about 1.5×10⁴ CFUs, about 1.5×10⁵ CFUs, about 1.5×10⁶ CFUs, about 1.5×10⁷ CFUs, about 1.5×10⁸ CFUs, about 1.5×10⁹ CFUs, or about 1.5×10¹⁰ CFUs, or any range comprising these values as endpoints), and/or an isolated population of Bacteroides vulgatus bacterial cells in an amount of between about 1.5×10⁴ and 1.5×10¹⁰ CFU (e.g., about 1.5×10⁴ CFUs, about 1.5×10⁵ CFUs, about 1.5×10⁶ CFUs, about 1.5×10⁷ CFUs, about 1.5×10⁸ CFUs, about 1.5×10⁹ CFUs, or about 1.5×10¹⁰ CFUs, or any range comprising these values as endpoints) (and any combination of amounts of Bacteroides ovatus, Bacteroides intestinalis, and/or Bacteroides vulgatus). In various aspects, the ratio of the isolated population of Bacteroides ovatus to the isolated population of Bacteroides intestinalis to the isolated population of Bacteroides vulgatus in a dose ranges from about 10:1:1 to about 1:10:1 to about 1:1:10 CFU. Optionally, the ratio of the isolated population of Bacteroides ovatus to the isolated population of Bacteroides intestinalis to the isolated population of Bacteroides vulgatus is about 1:1:1 CFU.

Various aspects of the disclosure are provided below:
Aspect 1. A composition comprising: (i) an isolated population of Bacteroides ovatus bacterial cells comprising three or more different Bacteroides ovatus subspecies; (ii) an isolated population of Bacteroides intestinalis bacterial cells comprising two or more different Bacteroides intestinalis subspecies; and (iii) an isolated population of Bacteroides vulgatus bacterial cells.
Aspect 2. The composition of aspect 1, wherein the isolated population of Bacteroides ovatus bacterial cells comprises Bacteroides ovatus M15A, Bacteroides ovatus M1B7, and Bacteroides ovatus M2H3.
Aspect 3. The composition of aspect 1 or 2, wherein the isolated population of Bacteroides intestinalis bacterial cells comprises Bacteroides intestinalis M2C7 and Bacteroides intestinalis M2F3.
Aspect 4. The composition of any one of aspect 1 to 3, wherein the isolated population of Bacteroides vulgatus comprises Bacteroides vulgatus M2E6.
Aspect 5. The composition of any one of aspect 1 to 4, wherein the composition comprises between 1×10¹ and 1×10¹⁰ colony forming units (CFU) of bacteria.
Aspect 6. The composition of any one of aspect 1 to 5, wherein the isolated population of Bacteroides ovatus bacterial cells comprises between about 1.5×10⁴ and 1.5×10¹⁰ colony forming units (CFU).
Aspect 7. The composition of any one of aspect 1 to 6, wherein the isolated population of Bacteroides intestinalis bacterial cells comprises between about 1.5×10⁴ and 1.5×10¹⁰ colony forming units (CFU).
Aspect 8. The composition of any one of aspect 1 to 7, wherein the isolated population of Bacteroides vulgatus bacterial cells comprises between about 1.5×10⁴ and 1.5×10¹⁰ colony forming units (CFU).
Aspect 9. The composition of any one of aspect 1 to 8, wherein the ratio of the isolated population of Bacteroides ovatus to the isolated population of Bacteroides intestinalis to the isolated population of Bacteroides vulgatus ranges from about 10:1:1 to about 1:10:1 to about 1:1:10 CFU.
Aspect 10. The composition of any one of aspect 1 to 8, wherein the ratio of the isolated population of Bacteroides ovatus to the isolated population of Bacteroides intestinalis to the isolated population of Bacteroides vulgatus is about 1:1:1 CFU.
Aspect 11. The composition of any one of aspect 1 to 10 further comprising one or more pharmaceutically acceptable excipients.
Aspect 12. The composition of any one of aspect 1 to 11, wherein the composition does not contain fecal matter.
Aspect 13. The composition for any one of aspect 1 to 11, wherein the composition comprises fecal matter.
Aspect 14. A method for increasing interferon (IFN) secretion in a subject, the method comprising administering to the subject the composition of any one of aspect 1 to 13.
Aspect 15. The method of aspect 14, wherein the subject is a mammal, optionally a human.
Aspect 16. The method of aspect 14 or 15, wherein the administration comprises oral administration.
Aspect 17. The method of aspect 14 or 15, wherein the administration comprises fecal transplantation.
Aspect 18. The method of any one of aspect 14 to 17, wherein the subject has or is suspected of having cancer.
Aspect 19. The method of aspect 18, wherein the cancer is non-small cell lung carcinoma (NSCLC).
Aspect 20. The method of any one of aspect 14 to 19, further comprising administering an immune checkpoint inhibitor (ICI) to the subject.
Aspect 21. The method of aspect 20, wherein the ICI comprises an anti-PD-1 antibody.
Aspect 22. A method for treating cancer in a subject, the method comprising administering to the subject the composition of any one of aspect 1 to 13.
Aspect 23. The method of aspect 22, wherein the subject is a mammal, optionally a human.
Aspect 24. The method of aspect 22 or 23, wherein the administration comprises oral administration.
Aspect 25. The method of aspect 22 or 23, wherein the administration comprises fecal transplantation.
Aspect 26. The method of any one of aspect 22 to 25, the cancer is non-small cell lung carcinoma (NSCLC).
Aspect 27. The method of any one of aspect 22 to 26, further comprising administering an immune checkpoint inhibitor (ICI) to the subject.
Aspect 28. The method of aspect 27, wherein the ICI comprises an anti-PD-1 antibody.
Aspect 29. The method of any one of aspect 22 to 28, wherein after the administration of the composition, interferon (IFN) secretion is increased in the subject relative to IFN secretion before the administration.
Aspect 30. The method of any one of aspect 14 to 29, wherein after administration of the composition, the isolated populations of Bacteroides ovatus, Bacteroides intestinalis, and Bacteroides vulgatus colonize the subject.

### EXAMPLES

### Example 1

The impact of bacteria on therapeutics is wide and includes modulation of chemotherapeutic and immunotherapeutic agents'efficacy and toxicity via metabolic and immune-mediated mechanisms. Intestinal microbiota profoundly impacts cancer patients' responses to ICI therapy, and microbial phylogeny is a poor predictor of anti-PD-1 anti-tumor response. Rather, microbial gene content may better capture the relationship between bacterial and ICI responsiveness. To study the relationship between intestinal microbiota and anti-PD-1 efficacy, pre-treatment (baseline) stool samples were obtained from 64 stage III/IV non-small cell lung carcinoma (NSCLC) patients undergoing PD-1 therapy. These patients were categorized as “Responders” or “Non-Responders” using RECIST criteria. It was observed that Responder patients have both a different microbial community structure than Non-Responders (P=0.0043), and a different bacterial transcriptome (PC2=0.03).

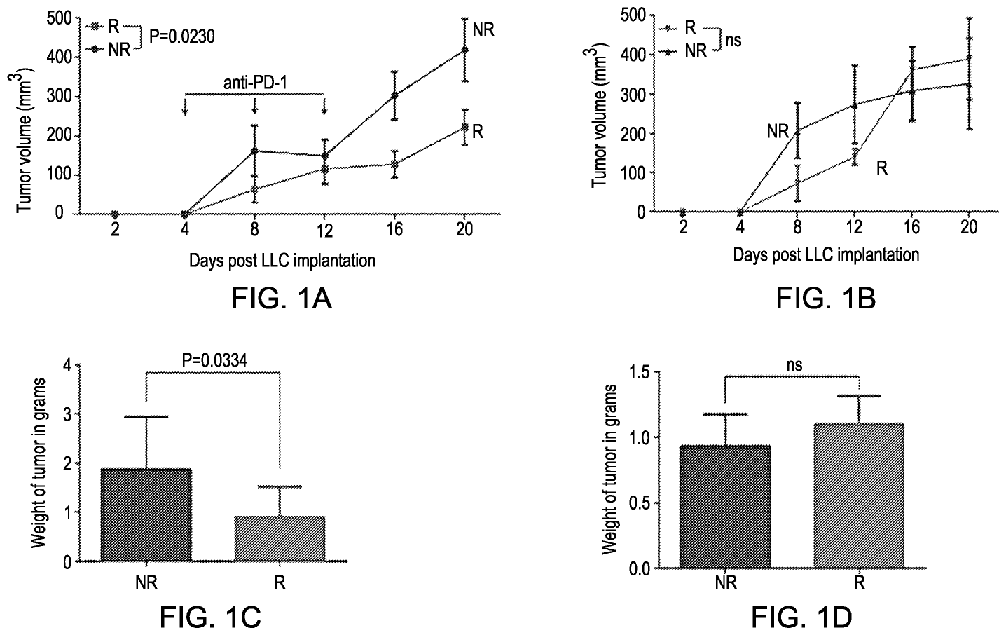

A Lewis Lung Carcinoma (LLC) syngeneic xenograft gnotobiotic mouse model and fecal microbiota transplantation (FMT) approach were used to functionally test anti-PD-1 Responder microbiota. Data indicate a significant decrease in tumor growth and weight in mice colonized with Responder microbiota compared to mice colonized with Non-Responder human feces (FIGS. 1A-1D). In the NSCLC cohort, a higher relative abundance of Ruminococcaceae (genus Ruminococcus and Faecalibacterium) were identified in the feces of PD-1 responding (R) compared to non-responding (NR) patients. Since the syngeneic xenografts were devoid of intratumor bacteria, it is likely that intestinal microbiota exerts synergy with ICI through a systemic, long-distance effect from intestinal biota. It was observed that intestinal microbial-derived metabolites synergize with immune cells to promote anti-PD-1 anti-cancer effect.

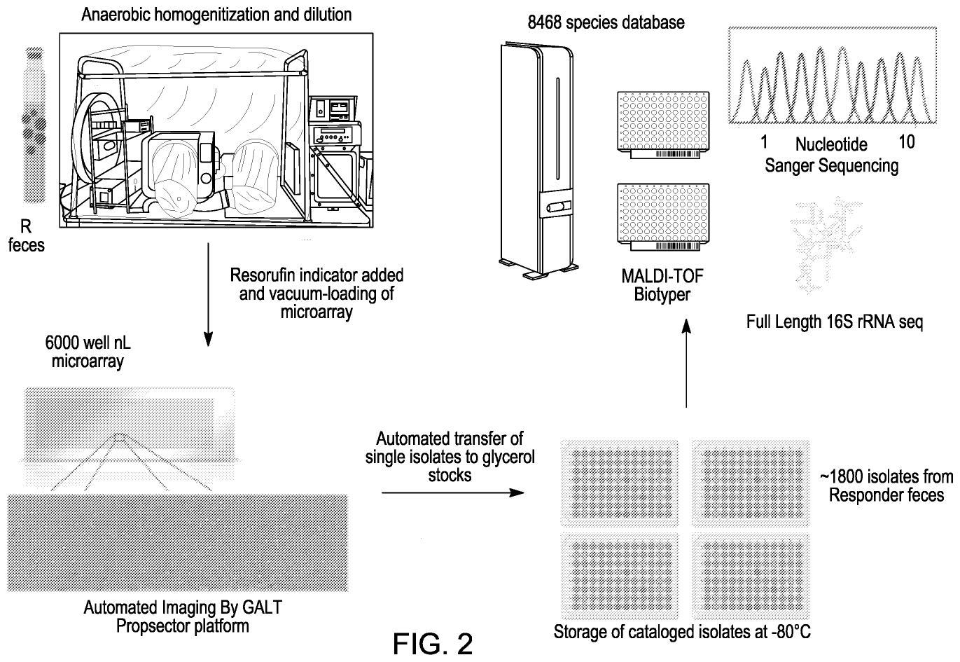

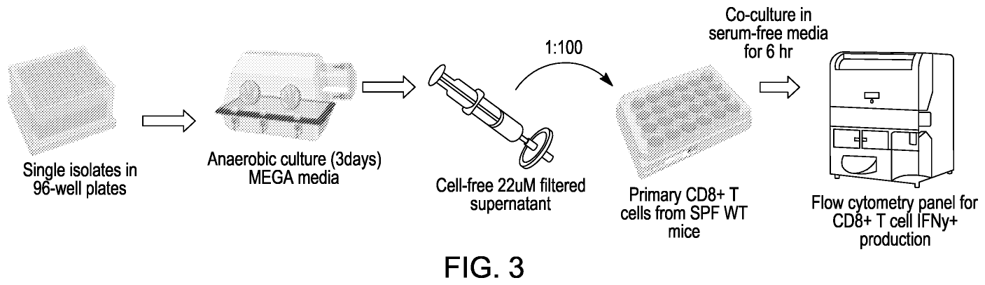

Next, high-throughput microbial isolation from feces of R-associated mice was performed using a GALT Prospector. One hundred and eighty three (183) bacterial strains were identified using MALDI-TOF Biotyper and Sanger sequencing (FIG. 2). The immunomodulatory effect (e.g., interferon (IFN) secretion) of the cell free supernatant of individual cultures of each bacterium was tested on mouse splenic CD8⁺T cells (FIG. 3). Following this in vitro screen, six (6) bacterial strains with strong capacity to induce IFN secretion were identified: three (3) strains of Bacteroides ovatus, two (2) strains of Bacteroides intestinalis and one (1) strain of Bacteroides vulgatus. These microorganisms were combined into a consortium designated as six-consortium (also referenced herein as “R consortium” or “R-Cons” or “six-consort”).

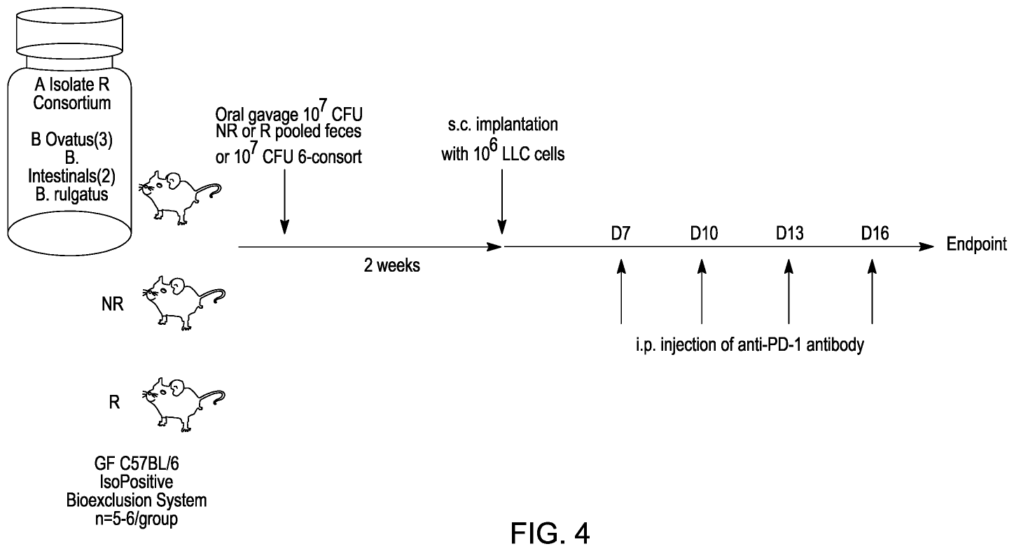

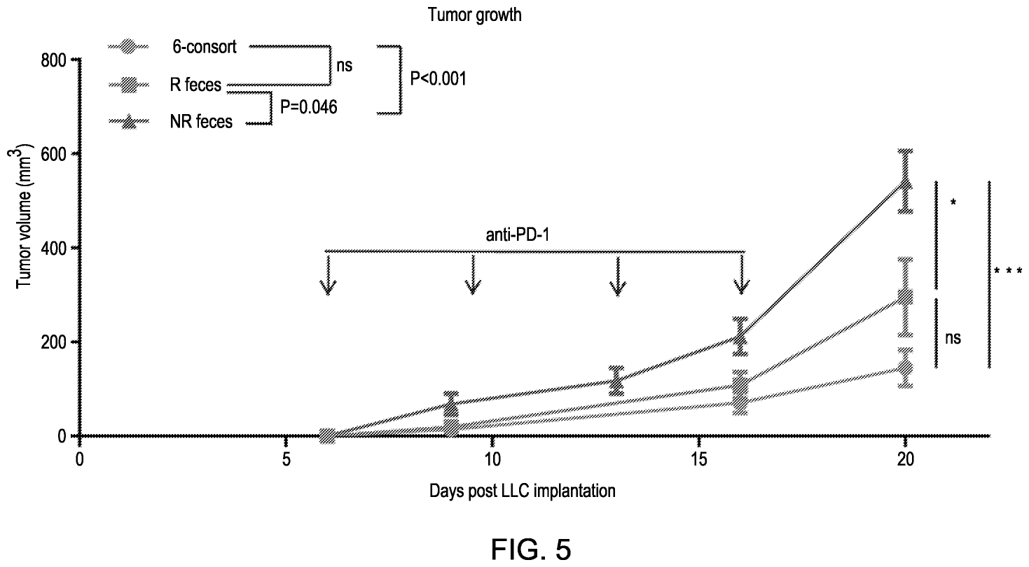

To test the effect of this consortium in anti-PD-1 mediated anti-tumor effect, germ-free mice were administered six-consortium (n=8), Responder feces (R-feces) (n=8), or NR feces (n=8) (FIG. 4). Two weeks after colonization, subcutaneous Lewis Lung Carcinoma (LLC) cells were implanted in the mice and once the tumor reached ˜20-30 mm, mice were treated with an anti-PD-1 antibody intraperitonally (i.p.). Four (4) injections were given every three (3) days. A significant decreased tumor growth in mice colonized with R-Cons compared to mice colonized with NR-feces was observed (FIG. 5). The anti-tumor effect of the six-consort was as efficient as feces from responding patients (R-feces). These findings indicate that the six-consortium possesses a synergistic effect with anti-PD-1 treatment and may enhance PD-1 responsiveness in patients with NSCLC.

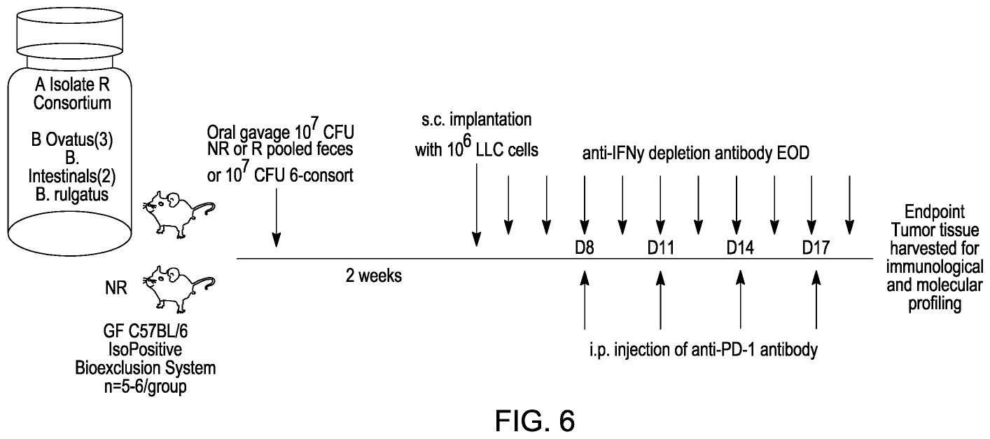

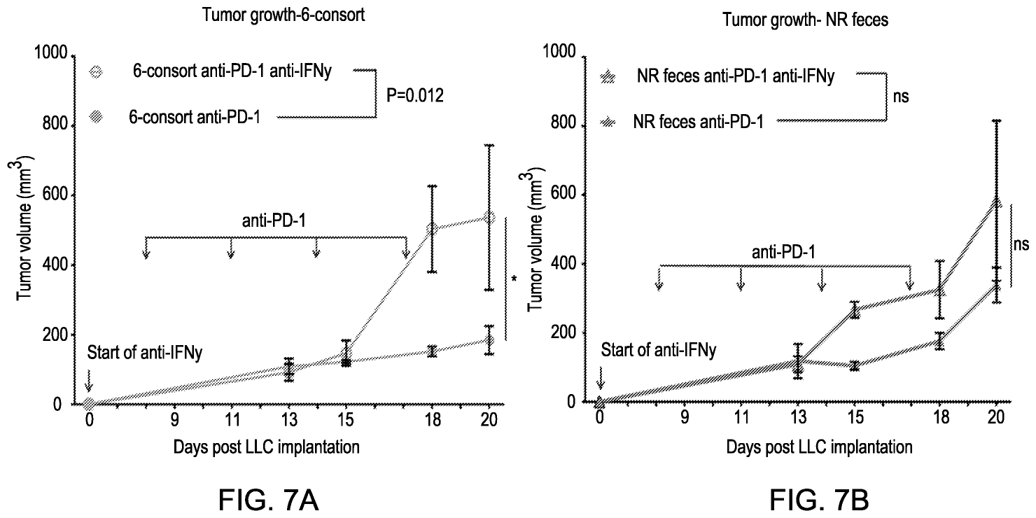

The effects of IFNγ depletion on the anti-tumor effects of R-Cons were investigated. FIG. 6 shows a schematic depicting testing the effect of anti-IFNγ depletion on six-consortium anti-PD-1 mediated anti-tumor effect. Briefly, germ-free mice were colonized with 1×10⁷ CFU of six-consortium or NR feces for 2 weeks followed by implantation with 1×10⁶ Lewis Lung Carcinoma (LLC) xenograft tumor cells. Mice then underwent four rounds of anti-PD-1 therapy with or without every other day anti-IFNγ therapy, both by intraperitoneal injection, with tumors being measured every three days by manual caliper. At endpoint, tumors were harvested for flow cytometric analysis. Data indicate in vivo depletion of IFNγ abrogates the beneficial effect of responder consortium on anti-PD-1. FIG. 7 shows a growth curve of LLC-luc subcutaneous xenograft tumors after human fecal microbiota transplant from Responder Consortium (RC; also referenced herein as “six-consort”') or Non-Responder (NR) pooled feces into germ-free mice treated with anti-PD-1 or combination anti-PD-1/anti-IFNγ monoclonal antibody injection. No significant difference between NR feces and NR feces with anti-IFNγ depletion was observed.

Previous reports suggested that the presence of bacteria in solid tumors could drive anti-tumor immunity, a process that could alter tumor immune environment and anti-PD-1 efficacy. Given this possibility, tumors were tested to determine if bacteria translocated to the tumor site. The bacterial 16S rRNA gene was not detected in tumors from humanized R and NR mice as assayed by PCR and qPCR, whereas a clear signal was observed in the feces of these mice. Thus, the responder biota mediates their synergistic effect through a long-distance mechanism.

### Example 2

Non-small cell lung cancer (NSCLC) patients that respond (R) to immune checkpoint blockade (ICB) have a different microbial community structure than non-responders (NR) pre-treatment. Example 1 describes that pooled R microbiota transplantation into gnotobiotic xenograft mice decreased tumor growth compared to NR colonized mice following anti-PD-1 therapy, and this decrease is associated with enrichment of the Bacteroides genus. This example describes isolation and identification of cell-free bacterial compositions (e.g., supernatants) containing bioactive molecules that are effective in reducing tumor growth.

Feces collected from R mice were used as source material for high-throughput microbial isolation performed with the GALT Prospector technology. Bacterial identification was performed using MALDI-TOF Biotyper and Sanger sequencing. The cell free supernatants and <3 kDa small molecules of 183 Bacteroides isolates were screened for their ability to stimulate IFNγ production via a bioassay using primary CD8+T cells and flow cytometric analysis. A consortium composed of six IFNγ-stimulating isolates or NR feces was transplanted into a gnotobiotic mouse model of lung cancer and treated with anti-PD-1, with or without anti-IFNγ monoclonal antibody depletion. Tumors were harvested at endpoint for flow cytometric analysis, and blood serum for IFNγ ELISA. Vacuum and high-performance liquid chromatography were used to identify fractions from the cell free supernatant of a single stimulating Bacteroides isolate which stimulate IFNγ production.

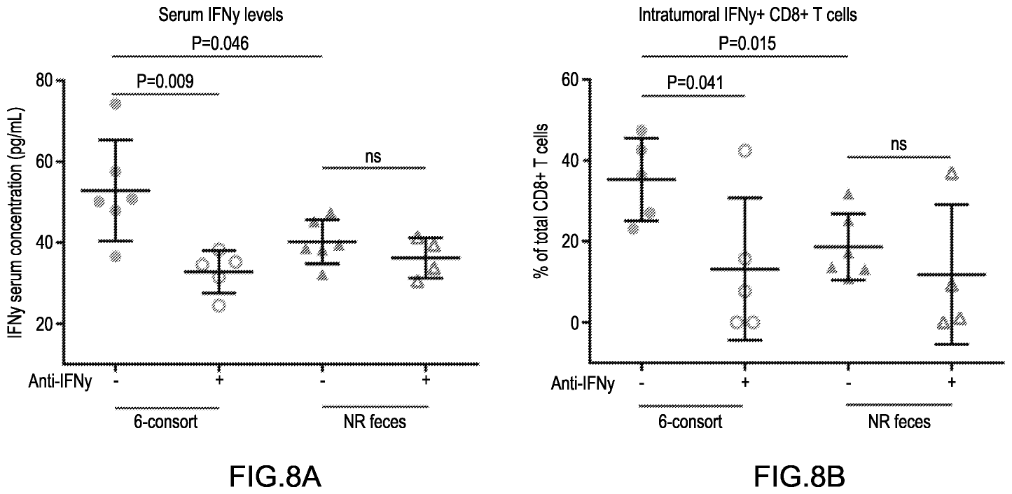

Six hundred and seventy nine (679) isolates from 30 unique species were cultured and identified. The cell free supernatant from six out of the 183 Bacteroides isolates stimulated IFNγ production from primary CD8+T cells. Small molecules from the combination of the six stimulatory isolates' supernatant significantly induced IFNγ production in CD8+T cells compared to 6 taxonomy-matched non-stimulatory isolates (P=0.039). A defined consortium composed of the six stimulatory isolates (six-consortium) was able to colonize germ free mice, and decreased tumor growth compared to NR feces-colonized mice (P=0.041). IFNγ depletion of anti-PD-1-treated six-consortium-treated mice significantly increased tumor growth (P=0.012) compared to non-depleted mice. Intratumor IFNγ+CD8+T cell frequency and circulating serum IFNγ was elevated only in tumors of six-consortium-treated mice. See FIGS. 8A-8B.

A microbial consortium engineered from R patients' feces synergize with anti-PD-1 therapy to reduce lung cancer growth through an IFNγ-dependent mechanism which may be mediated by small molecule metabolites.

### Example 3

This example provides a method for generating MALDI TOF spectra for characterizing bacteria (e.g., for characterizing Bacteroides ovatus M15A (FIG. 9A), Bacteroides ovatus M1B7 (FIG. 9B), Bacteroides ovatus M2H3 (FIG. 9C), Bacteroides intestinalis M2C7 (FIG. 9D), Bacteroides intestinalis M2F3 (FIG. 9E), and Bacteroides vulgatus M2E6 (FIG. 9F)).

Unique bacterial isolates were cultured from responder feces using the Isolation Bio Prospector platform coupled with identification via MALDI Biotyper®/full length 16S rRNA sanger sequencing. The fecal sample was aliquoted and homogenized in anaerobic MEGA medium (Han et al., Nature, 595 (7867) (2021), 415-420) to a final suspension of 10% (w/v). To estimate microbial load of initial responder (R) mouse fecal sample, a dilution to extinction method was used by performing serial dilutions of the original sample and observing anaerobic growth over 48 hours. The fecal sample was then diluted to a theoretical target loading of 0.3 cells/well in addition to 50 μM of resorufin as an anaerobic growth indicator. The diluted sample was then vacuum loaded onto 6000 nanowell growth chambers, sealed, and imaged using the Isolation Bio Prospector automated imaging platform. Following anaerobic incubation for 2-5 days, isolates were identified via subtractive imaging of green fluorescence. The Isolation Bio Prospector identified wells likely to contain culturable, single bacterial isolates, and performed sterile transfers from the array into 96 well plates from which glycerol stocks were prepared, cataloged, and stored at −80 in sealed anaerobic gas packs. To identify individual isolates, 96 well plates of isolates were cultured using 10 μL of original glycerol stock into 190 μL MEGA media for 2-5 days, and then pelleted via centrifugation. Each bacterial growth pellet was streaked in duplicate onto a stainless-steel reusable MALDI Biotyper® (Bruker) target plate and overlaid with HCCA matrix (Bruker) and 70% formic acid. The dried target plates were then run and analyzed for species identification on the Bruker MALDI Biotyper® Sirius system in the University of Florida Interdisciplinary Center for Biotechnology Research Proteomics Core Facility. The spectra resulting from the strains are set forth in FIGS. 9A-9F and uniquely identify the bacteria consortium described herein.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context; the terms “a” (or “an”), “one or more,” and “at least one” can be used interchangeably herein. The term “or” should be understood to encompass items in the alternative or together, unless context unambiguously requires otherwise. The term “and/or” should be understood to encompass each item in a list (individually), any combination of items a list, and all items in a list together. The terms “comprising,” “having,” “including,” and “containing” are to be construed as open-ended terms (i.e., meaning “including, but not limited to,”) unless otherwise noted. The disclosure contemplates embodiments described as “comprising” a feature to include embodiments which “consist of” or “consist essentially of” the feature. Other than in the operating examples, or where otherwise indicated, all numbers expressing quantities of ingredients or reaction conditions used herein should be understood as modified in all instances by the term “about” as that term would be interpreted by the person skilled in the relevant art. The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviation, per the practice in the art. Alternatively, “about” can mean a range of up to 10%, up to 5%, or up to 1% of a given value.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein. In any of the ranges described herein, the endpoints of the range are included in the range. However, the description also contemplates the same ranges in which the lower and/or the higher endpoint is excluded.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

What is claimed:

1. A composition comprising:
(i) an isolated population of Bacteroides ovatus bacterial cells comprising three or more different Bacteroides ovatus subspecies;
(ii) an isolated population of Bacteroides intestinalis bacterial cells comprising two or more different Bacteroides intestinalis subspecies; and
(iii) an isolated population of Bacteroides vulgatus bacterial cells.

2. The composition of claim 1, wherein the isolated population of Bacteroides ovatus bacterial cells comprises Bacteroides ovatus M15A, Bacteroides ovatus M1B7, and Bacteroides ovatus M2H3.

3. The composition of claim 1 or 2, wherein the isolated population of Bacteroides intestinalis bacterial cells comprises Bacteroides intestinalis M2C7 and Bacteroides intestinalis M2F3.

4. The composition of any one of claims 1 to 3, wherein the isolated population of Bacteroides vulgatus comprises Bacteroides vulgatus M2E6.

5. The composition of any one of claims 1 to 4, wherein the composition comprises between 1×10¹ and 1×10¹⁰ colony forming units (CFU) of bacteria.

6. The composition of any one of claims 1 to 5, wherein the isolated population of Bacteroides ovatus bacterial cells comprises between about 1.5×10⁴ and 1.5×10¹⁰ colony forming units (CFU).

7. The composition of any one of claims 1 to 6, wherein the isolated population of Bacteroides intestinalis bacterial cells comprises between about 1.5×10⁴ and 1.5×10¹⁰ colony forming units (CFU).

8. The composition of any one of claims 1 to 7, wherein the isolated population of Bacteroides vulgatus bacterial cells comprises between about 1.5×10⁴ and 1.5×10¹⁰ colony forming units (CFU).

9. The composition of any one of claims 1 to 8, wherein the ratio of the isolated population of Bacteroides ovatus to the isolated population of Bacteroides intestinalis to the isolated population of Bacteroides vulgatus ranges from about 10:1:1 to about 1:10:1 to about 1:1:10 CFU.

10. The composition of any one of claims 1 to 8, wherein the ratio of the isolated population of Bacteroides ovatus to the isolated population of Bacteroides intestinalis to the isolated population of Bacteroides vulgatus is about 1:1:1 CFU.

11. The composition of any one of claims 1 to 10 further comprising one or more pharmaceutically acceptable excipients.

12. The composition of any one of claims 1 to 11, wherein the composition does not contain fecal matter.

13. The composition for any one of claims 1 to 11, wherein the composition comprises fecal matter.

14. A method for increasing interferon (IFN) secretion in a subject, the method comprising administering to the subject the composition of any one of claims 1 to 13.

15. The method of claim 14, wherein the subject is a mammal, optionally a human.

16. The method of claim 14 or 15, wherein the administration comprises oral administration.

17. The method of claim 14 or 15, wherein the administration comprises fecal transplantation.

18. £ The method of any one of claims 14 to 17, wherein the subject has or is suspected of having cancer.

19. The method of claim 18, wherein the cancer is non-small cell lung carcinoma (NSCLC).

20. The method of any one of claims 14 to 19, further comprising administering an immune checkpoint inhibitor (ICI) to the subject.

21. The method of claim 20, wherein the ICI comprises an anti-PD-1 antibody.

22. A method for treating cancer in a subject, the method comprising administering to the subject the composition of any one of claims 1 to 13.

23. The method of claim 22, wherein the subject is a mammal, optionally a human.

24. The method of claim 22 or 23, wherein the administration comprises oral administration.

25. The method of claim 22 or 23, wherein the administration comprises fecal transplantation.

26. The method of any one of claims 22 to 25, the cancer is non-small cell lung carcinoma (NSCLC).

27. The method of any one of claims 22 to 26, further comprising administering an immune checkpoint inhibitor (ICI) to the subject.

28. The method of claim 27, wherein the ICI comprises an anti-PD-1 antibody.

29. The method of any one of claims 22 to 28, wherein after the administration of the composition, interferon (IFN) secretion is increased in the subject relative to IFN secretion before the administration.

30. The method of any one of claims 14 to 29, wherein after administration of the composition, the isolated populations of Bacteroides ovatus, Bacteroides intestinalis, and Bacteroides vulgatus colonize the subject.

31. A method for increasing interferon (IFN) secretion in a subject, the method comprising administering to the subject a composition comprising Bacteroides ovatus bacterial cells, Bacteroides intestinalis bacterial cells, Bacteroides vulgatus bacterial cells, or a combination thereof.

32. A method for treating cancer in a subject in need thereof, the method comprising administering to the subject a composition comprising Bacteroides ovatus bacterial cells, Bacteroides intestinalis bacterial cells, Bacteroides vulgatus bacterial cells, or a combination thereof.

33. The method of claim 31 or claim 32, wherein the composition comprises Bacteroides ovatus M15A bacterial cells, Bacteroides ovatus M1B7 bacterial cells, Bacteroides ovatus M2H3 bacterial cells, or a combination thereof.

34. The method of any one of claims 31 to 33, wherein the composition comprises Bacteroides intestinalis M2C7 bacterial cells, Bacteroides intestinalis M2F3 bacterial cells, or a combination thereof.

35. The method of any one of claims 31 to 34, wherein the isolated population of Bacteroides vulgatus comprises Bacteroides vulgatus M2E6 bacterial cells.

36. The method of claim 31 or claim 32, wherein the composition comprises Bacteroides ovatus M2H3 cells bacterial cells.

37. The method of any one of claims 32 to 36, wherein the cancer is non-small cell lung carcinoma (NSCLC).

38. The method of any one of claims 32 to 37, further comprising administering an immune checkpoint inhibitor (ICI) to the subject.

39. The method of claim 38, wherein the ICI is an anti-PD-1 antibody.
